(19) United States
(12) Patent Application Publication
(10) Pub. No.: US 2004/0241724 A1
(43) Pub. Date: Dec. 2, 2004

(54) Immobilization method and kit therefor

(75) Inventors: Robert Karlsson, Uppsala (SE); Anders Sjodin, Uppsala (SE)

Correspondence Address: SEED INTELLECTUAL PROPERTY LAW GROUP PLLC, 701 FIFTH AVE, SUITE 6300, SEATTLE, WA, 98104-7092, US

(73) Assignee: Biacore AB, Uppsala (SE)

(21) Appl. No.: 10/806,028

(22) Filed: Mar. 22, 2004

Related U.S. Application Data
(60) Provisional application No. 60/457,508, filed on Mar. 25, 2003.

(30) Foreign Application Priority Data
Mar. 25, 2003 (SE) 0300805-9

(51) Int. Cl. C12Q 1/68; C12M 1/34; B05D 3/00

(52) U.S. Cl. 435/6; 435/287.2; 427/2.11

(57) ABSTRACT

A method of immobilizing a target molecule to a solid support surface capable of interacting with the target molecule is disclosed. The method comprises the steps of complexing the target molecule with a vesicular structure capable of forming a dissociable complex with the target molecule, contacting the complex formed with the solid support surface to thereby bind the target molecule to the surface, dissociating the complex, and removing the vesicular structure from the solid support surface to leave the target molecule immobilized on the surface. A method of sensitizing a solid support surface, the use of the sensitized solid support surface for analyzing analytes, and a reagent kit for carrying out the method are also disclosed.

### CROSS-REFERENCES TO RELATED APPLICATIONS

This application claims the benefit of U.S. Provisional Application No. 60/457,508 filed Mar. 25, 2003, and also claims priority to Swedish Application No. 0300805-9 filed Mar. 25, 2003, both of which applications are incorporated herein by reference in their entirety.

### BACKGROUND OF THE INVENTION

1. Field of the Invention

The present invention relates to the preparation of solid support surfaces, and more particularly to a method of immobilizing target molecules to solid support surfaces. The invention also relates to a method of sensitizing a sensor surface, to the use of the sensitized sensor surface for analyzing analytes, and to a reagent kit for carrying out the method.

2. Description of the Related Art

A variety of analytical techniques are used to characterize interactions between molecules, particularly in the context of assays directed to the detection and interaction of biomolecules. For example, antibody-antigen interactions are of fundamental importance in many fields, including biology, immunology and pharmacology. In this context, many analytical techniques involve binding of a “ligand”, such as an antibody, to a solid support, followed by contacting the ligand with an “analyte”, such as an antigen. Following contact of the ligand and analyte, some characteristic is measured which is indicative of the interaction, such as the ability of the ligand to bind the analyte. It is often desired that after measurement of the interaction, it should be possible to dissociate the ligand-analyte pair in order to “regenerate” free ligand, thereby enabling reuse of the ligand surface for a further analytical measurement.

The binding, or immobilization, of the ligand to the support can be either direct or indirect. In direct immobilization, the ligand is coupled directly to the surface, typically covalently, whereas in indirect immobilization the ligand is captured (usually by non-covalent binding) by a molecule that is directly coupled to the surface, typically covalently. While indirect immobilization is restricted to ligands that have a suitable binding site or tag for the surface-coupled molecule, it has several advantages. For example, non-covalent immobilization of the ligand is simpler to effect than covalent coupling, and the ligand need not be pure but can be captured from a crude sample. Further, all ligands are immobilized in a known and consistent orientation on the surface. Usually, it is also possible to regenerate the ligand surface by general regeneration conditions so that repeated capture can be performed on the surface. Exemplary capture molecules include protein A and protein G which both bind to the Fc-part of immunoglobulins (antibodies), NTA metal chelates which bind to histidine tags, and oligonucleotides which hybridize to a complementary oligonucleotide tag.

The use of oligonucleotides as capture molecules, which is disclosed in more detail in, e.g., U.S. Pat. No. 5,648,213, has the additional advantage that by varying the oligonucleotide sequences very specific oligonucleotide pairs may easily be created. The specificity of capture oligonucleotides bound to discrete surface areas, or spots, on a solid support can then be used to address different ligands to the different spots. Another advantage resides in that relatively harsh conditions may be used to regenerate the oligonucleotide capture surface as compared to, for example, a protein surface which is susceptible to denaturation. A problem in immobilizing oligonucleotides to a solid support, however, is their relatively large negative charge, which makes it difficult to immobilize them to negatively charged surfaces. Traditional approaches to overcome this problem, including the use of high salt concentrations to shield off the charges, or low pH conditions, have either failed or given low immobilization degrees, as will be demonstrated in comparative examples below.

In the genetic engineering field, it is previously known to use polycations as nonviral agents for delivering DNA to cells. The polycations efficiently bind to negatively charged DNA, which results in a substantial DNA compaction. Also, if the polyelectrolyte complex formed has an overall positive charge, it increases the interaction with a negatively charged cell membrane. The DNA/polyelectrolyte complex formed interacts with the cell surface, and the DNA is then taken up by the cell, probably through lysosomes or endosomes.

Positively-charged, neutral and negatively-charged liposomes as well as positively-charged micelles have also been used for the delivery of nucleic acids to cells, in the micelle case relying on the positive charge of the micelles to provide a “cross-bridge” between the polyanionic nucleic acids and the polyanionic surfaces of the cells. For example, U.S. Pat. No. 6,210,717 discloses the use of a mixed polymeric micelle containing an amphiphatic polyester-polycation copolymer and an amphiphatic polyester-sugar copolymer to deliver a selected nucleic acid into a host cell.

It is an object of the present invention to overcome the problem of immobilizing oligonucleotides to a negatively charged surface.

### BRIEF SUMMARY OF THE INVENTION

The above and other objects and advantages are provided by a novel method for immobilizing a target molecule to solid support surface wherein the target molecule is first complexed with a vesicular structure, whereupon the complex is contacted with the solid support to permit the target molecule to bind to the surface.

In one aspect, the present invention therefore provides a method of immobilizing a target molecule to a solid support surface capable of interacting with the target molecule, which method comprises the steps of:

complexing the target molecule with a vesicular structure capable of forming a dissociable complex with the target molecule,

contacting the complex formed with the solid support surface to thereby bind the target molecule to the surface and dissociate the complex, and

removing the vesicular structure from the solid support surface to leave the target molecule immobilized on the surface.

If the complex is not dissociated when binding to the solid support surface, a subsequent treatment or wash may be required.

In another aspect, the present invention provides a method of sensitizing a solid support surface with a ligand, which method comprises immobilizing to the surface a capture agent capable of binding the ligand according to the first method aspect above, and then contacting the surface with the ligand to bind the ligand to the immobilized capture agent.

In still other aspects, the present invention provides the use of a solid support surface prepared according to the first method aspect above for assaying a sample for at least one analyte which can bind to at least one ligand on the surface, or for studying interactions of at least one analyte with at least one ligand on the surface, respectively.

In yet another aspect, the present invention provides a reagent kit that may be used for carrying out the above-mentioned methods and uses of the invention.

The above and other aspects of the invention will be evident upon reference to the accompanying drawing and the following detailed description.

### BRIEF DESCRIPTION OF THE DRAWING

FIG. 1 is a schematic illustration of a micelle/oligonucleotide complex.

### DEFINITIONS

In describing and claiming the present invention, the following terminology will be used in accordance with the definitions set out below.

“Solid support” refers to any solid (flexible or rigid) substrate onto which it is desired to immobilize one or more target compounds. The substrate may be biological, non-biological, organic, inorganic or a combination thereof, and may be in the form of particles, strands, precipitates, gels, sheets, tubings, spheres, containers, capillaries, pads, slices, films, plates, slides, etc, having any convenient shape, including disc, sphere, circle, etc.

“Complex” refers to a chemical association of two (or more) species joined usually by weak electrostatic forces. A complex is dissociable if it can be dissociated into the two complex-forming species.

“Vesicular structure” refers to an organized structure of amphiphatic (surfactant) molecules having both hydrophobic and hydrophilic domains, and includes, for example, liposomes, micelles or inverse micelles. Liposomes are spherical vesicles formed by a bilayer of lipids, usually phospholipids, that enclose an aqueous volume, whereas micelles and inverse micelles are colloidal aggregates of amphiphatic molecules, which occur at (and above) a well-defined concentration known as the critical micelle concentration (CMC). The typical number of aggregated molecules in a micelle is about 50 to 100.

“Functional group” refers to a reactive chemical entity. When present on a solid support surface, the functional group serves to connect a binding agent, such as a capture agent or a ligand, to the surface. Usually, functional groups need to be activated in order to immobilize a binding agent. The functional groups may be inherently present in the material used for the solid support or they may be provided by treating or coating the support with a suitable material containing the functional group. A functional group may also be introduced by reacting the solid support surface with an appropriate chemical agent.

“Activation” refers to a modification of a functional group to a reactive group enabling or improving coupling of a binding agent thereto.

“Ligand” refers to a molecule that has a known or unknown affinity for a given analyte. The ligand may be a naturally occurring molecule or one that has been synthesized. The ligand may be used per se or as aggregates with another species. Optionally, the ligand may also be a cell.

“Analyte” refers to a molecule, which may be a macromolecule, such as a polypeptide or a polynucleotide, or even a small molecule, the presence, amount and/or identity of which are to be determined, or which is to be characterized in other respects, such as, e.g., its binding properties. The analyte is recognized by a particular ligand forming an analyte/ligand pair. Optionally, the ligand may also be a cell. As used herein, the term analyte also includes analyte analogues.

“Capture agent” refers to a binding agent that can be immobilized to a solid support surface and which can bind to another species, such as a ligand or a second capture agent.

“Specific binding pair”(abbreviated “sbp”) refers to a pair of molecules (each being a member of a specific binding pair) that are naturally derived or synthetically produced. One of the pair of molecules has a structure (such as an area or cavity) on its surface that specifically binds to (and is therefore defined as complementary with) a particular structure (such as a spatial and polar organisation) of the other molecule, so that the pair have the property of binding specifically to each other. Examples of types of specific binding pairs are protein-protein, antigen-antibody, antibody-hapten, biotin-avidin, ligand-receptor (e.g., hormone receptor, peptide-receptor, enzyme-receptor), carbohydrate-protein, carbohydrate-lipid, lectin-carbohydrate, nucleic acid-nucleic acid (including, e.g., PNA-PNA; PNA=peptide nucleic acid), histidine residue(s)-metal chelate.

“Antibody” refers to an immunoglobulin which may be natural or partly or wholly synthetically produced and also includes active fragments, including Fab antigen-binding fragments, univalent fragments and bivalent fragments. The term also covers any protein having a binding domain that is homologous to an immunoglobulin binding domain. Such proteins can be derived from natural sources, or partly or wholly synthetically produced. Exemplary antibodies are the immunoglobulin isotypes and the Fab, Fab′, F(ab′)₂, scFv, Fv, dAb, and Fd fragments.

“Nucleic acid” refers to a deoxyribonucleotide polymer (DNA) or ribonucleotide polymer (RNA) in either single-or double-stranded form, and also encompasses synthetically produced analogs that can function in a similar manner as naturally occurring nucleic acids. While natural nucleic acids have a phosphate backbone, artificial nucleic acids may contain other types of backbones, nucleotides or bases. These include, for instance, peptide nucleic acids (PNAs) as described in, e.g., U.S. Pat. No. 5,948,902 and the references cited therein; pyranosyl nucleic acids (p-NAs) as described in, e.g., WO 99/15540 (p-RNAs), WO 99/15539 (p-RNAs), and WO 00/11011 (p-DNAs); locked nucleic acids (LNAs), as described in, e.g., U.S. Pat. No. 6,316,198; and phosphothionates and other variants of the phosphate backbone of native nucleic acids.

“Oligonucleotide” refers to single stranded nucleotide multimers of from about 5 to about 100 nucleotides (including synthetically produced analogs as described for “nucleic acid” above).

“Polynucleotide” refers to single stranded nucleotide multimers of from about 100 nucleotides (including synthetically produced analogs as described for “nucleic acid” above).

“Low molecular weight organic compound” refers to an organic compound having a molecular weight in the range of from about 100 to about 1000, usually from about 250 to about 800. Low molecular weight compounds are sometimes also referred to as “small molecules”.

“Array” generally refers to a linear or two-dimensional array of discrete regions, each having a finite area, formed on a continuous surface of a solid support and supporting one or more binding agents, such as, e.g., capture agents or ligands. Ordered arrays of nucleic acids, proteins, small molecules, cells or other substances on a solid support enable parallel analysis of complex biochemical samples. In a “microarray”, the density of discrete regions, or spots, is typically at least 100/cm², and the discrete regions typically have a diameter in the range of about 10-1000 μm, usually about 10-500 μm and are separated from other regions in the array by about the same distance.

“Biosensor” usually refers to a device that uses a component for molecular recognition (for example a layer with immobilised antibodies) in conjunction with a solid state physicochemical transducer.

In the specification and the appended claims, the singular forms “a”, “an”, and “the” are meant to include plural reference unless it is stated otherwise. Also, unless defined otherwise, technical and scientific terms used herein have the same meaning as commonly understood to a person skilled in the art related to the invention.

### DETAILED DESCRIPTION OF THE INVENTION

As mentioned above, the present invention generally relates to the immobilization of a target molecule to a solid support surface. Solid supports with immobilized target molecules are used in various fields, including analytical and separation techniques. Immobilization of a target molecule to a solid support often involves binding, such as covalent binding, of the molecule to the solid support surface. Usually, the solid support has a reactive chemical group that can react with the target molecule. Sometimes, the target molecule carries an electric charge, and if the solid support surface carries an opposite charge, the target molecule will be attracted to the solid support surface, facilitating the molecular interaction with the surface. On the other hand, if the solid support surface carries the same kind of charge as that of the target molecule, it is readily seen that immobilization may be reduced or even substantially prevented. An example of the latter case is the immobilization of oligonucleotides (which generally carry a negative charge) to negatively charged surfaces.

According to the present invention, it has now been found that a charged target molecule, particularly a macromolecule, such as, e.g., an oligonucleotide, may be efficiently immobilized to a solid support surface, even if the surface carries the same kind of charge as that of the target molecule, if the target molecule is first complexed with a vesicular structure carrying a charge opposite to that of the target molecule, and the complex formed is then contacted with the solid support surface.

Without being limited to any particular theory, it is assumed that the improved immobilization efficiency is due to the target molecule/vesicular structure complex permitting the target molecule to come sufficiently near the surface for it to interact therewith. This is in turn assumed to be due, on one hand, to the, e.g., positively charged vesicular structure at least partially neutralizing the opposite, e.g., negative, charge of the target molecule, and, on the other hand, e.g., in the case of a macromolecule such as an oligonucleotide, compacting of the macromolecule by the vesicular structure. Compacting may also result in faster diffusion of the molecule to the surface as compared to the diffusion of the free molecule. With regard to the assumed favorable compaction of a target molecule, the formation of the target molecule/vesicular structure complex may also improve immobilization thereof to a neutral solid support surface. Reducing repulsion forces between molecules to be immobilized may also lead to higher immobilization densities.

The present inventive concept is generally applicable to any target molecule that can form a dissociable complex with a charged or uncharged vesicular structure. The target molecule is, however, preferably a biomolecule (including synthetically produced biomolecules and analogues thereto), such as, e.g., nucleic acids (including, e.g., plasmids), proteins, polypeptides, lipids, and carbohydrates. More specific examples of biomolecules are oligonucleotides, polynucleotides, antibodies and enzymes. The target molecule may also be a low molecular weight organic compound.

The vesicular structure is preferably a liposome or a micelle, especially a micelle. Liposomes or micelles may be mixed liposomes or mixed micelles, i.e., containing two (or more) liposome and micelle forming components, respectively.

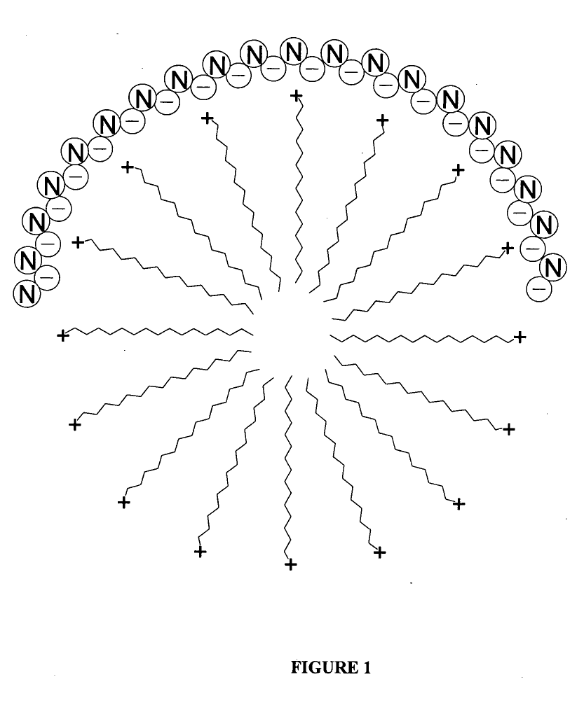

In one embodiment of the present invention, a nucleic acid, particularly a nucleotide, is immobilized to a negatively charged solid support surface using a positively charged micelle or liposome. A schematic illustration of a complex between an oligonucleotide (20-mer) and a positively charged micelle (CTAB) is shown in FIG. 1.

In another embodiment of the invention, a protein, particularly an antibody, carrying a negative charge is immobilized to a negatively charged solid support surface using a positively charged micelle or liposome.

In yet another embodiment of the invention, a low molecular weight organic compound, such as adenosine triphosphate (ATP) or nitrilo tri-acetic acid (NTA), carrying a negative charge is immobilized to a negatively charged solid support surface by means of a positively charged micelle or liposome.

In still another embodiment, a positively charged target molecule is immobilized to a positively charged solid support surface using a negatively charged micelle or liposome.

The relative amounts of target molecules and micelles or liposomes to be used depend on inter alia the particular target molecule and micelle or liposome, respectively, but suitable ratios may readily be established by a person skilled in the art. Generally, ratios of target molecule to micelle or liposome (number of target molecules to number of micelles) from about 1:3 to about 3:1, preferably from about from 1:2 to about 2:1, especially about 1:1 may be used.

The preparations of micelles and liposomes are well known to the skilled person and need not be described in any detail herein. For general descriptions of methods therefor it may be referred to, for example, Szoka, F., Jr., and Papahadjopoulos, D., Annu. Rev. Biophys. Bioeng. 9, 467 (1980); and Schwendener, R. A., Ansanger, M., and Weder, H. G., Biochem. Biophys. Res. Commun., 100, 1055 (1981) (the disclosures of which are incorporated by reference herein).

Examples of amphiphatic molecules, or surfactants, from which positively charged micelles may be prepared include dodecyltrimethylammonium bromide (DTAB), cetyltrimethylammonium bromide (CTAB) (other name: hexadecyltrimethylammonium bromide), benzyldimethylhexadecylammonium chloride, dimethyldioctadecylammonium bromide, dodecylethyldimethylammonium bromide, ethylhexadecyldimethylammonium bromide, trimethyl(tetradecyl)ammonium bromide, and thonzonium bromide.

Negatively charged micelles may, for example, be prepared from sodium dodecylsulphate (SDS).

Examples of molecules from which positively or negatively charged liposomes may be prepared include hexadecyldimethylammoniumpropane-1-sulphonate.

Usually, it is preferred that only a part of the electric charge of the micelle or liposome is neutralized by the target molecule, such that there will remain a residual charge after the complex formation to permit the complex to interact electrostatically with an oppositely charged solid support surface.

The complex between target molecule and the vesicular structure is usually formed spontaneously. When the complex is then contacted with the solid support, the target molecule interacts with the reactive functional group or other binding moiety on the surface, so that the target molecule is immobilized thereto. This may cause simultaneous dissociation of the complex. Usually, however, a treatment or wash with a suitable solution will be required to dissociate the complex and remove the vesicular structure or residues thereof from the surface. The necessary solutions/conditions therefor are either well known to or may readily be established by the skilled person.

The solid support is preferably a rigid structure and may comprise a substrate having a surface layer of a different material. While the solid support may be a particle, it is usually a surface of a larger entity, such as an inner surface of a well or receptacle, or a plate or slide. Exemplary of the latter kind are solid supports used for protein or DNA/RNA chips, as well as sensing surfaces in sensor devices, such as biosensors.

The surface of the solid support may be composed of a variety of materials, for example, polymers, plastics, resins, polysaccharides, silica or silica-based materials, carbon, metals, inorganic glasses, membranes, etc. A suitable surface is a metal film, e.g., gold, silver, or aluminum, preferably gold.

To permit immobilization of target molecules, the solid support surface comprises groups or molecules capable of interacting with the target molecule. Such groups may be functional groups, e.g., hydroxy, carboxy, amino, formyl, hydrazide, carbonyl, epoxy or vinyl, which may form a covalent bond with a functional group on the target molecule. Usually, the functional group(s) on the surface or on the target molecule is (are) activated to a more reactive group(s) prior to reaction with the target molecule. For example, an aminonucleotide may be coupled to a surface-bound carboxy group activated to a N-hydroxysuccinmide ester group. It is to be noted that a surface with an activated functional group that is, e.g., negatively charged before the activation but neutral after the activation may develop a negative charge during the immobilization process due to competing hydrolysis of the activated group.

Alternatively, the solid support surface carries one member of a specific binding pair (sbp), and the other member of the specific binding pair is on the target molecule. A commonly used sbp for immobilizing oligonucleotides is biotin-avidin (or streptavidin).

Especially in biosensor contexts, the solid support surface may be part of a flow cell, permitting the methods of the invention to be performed in situ in the flow cell. Examples of flow cells used in the biosensor field are described in, e.g., U.S. Pat. Nos. 5,492,840, 5,513,264 and WO 99/36766 (the relevant disclosures of which are incorporated by reference herein).

As mentioned above, the target molecule may, for instance, be an analyte-binding ligand, or a capture agent capable of (usually specifically) binding a ligand. Alternatively, the capture agent may bind a binding agent which in turn can bind a ligand. A single capture agent may be used to bind a single ligand to the surface, or to bind different ligands to different discrete areas of the surface to provide a ligand array. Alternatively, a ligand array may be formed by selectively binding different ligands to specific capture agents provided on respective discrete surface areas. Binding of a ligand to a solid support surface, such as a sensor surface, is often referred to as “sensitizing” the surface.

Examples of ligands include, without any limitation thereto, agonists and antagonists for cell membranes, toxins and venoms, viral epitopes, antigenic determinants, hormones and hormone receptors, steroids, peptides, enzymes, substrates, cofactors, drugs, lectins, sugars, oligonucleotides, oligosaccharides, proteins, glycoproteins, cells, cellular membranes, organelles, cellular receptors, vitamins, viral epitopes, and immunoglobulins, e.g., monoclonal and polyclonal antibodies.

Exemplary capture agents include nucleic acids and antibodies, especially oligonucleotides. A surface with a capture agent in the form of an oligonucleotide may, for example, be used to bind a ligand conjugated to a complementary oligonucleotide tag. In this way, different ligands may readily be immobilized to different discrete areas of the surface. As mentioned above, it is also possible to immobilize different oligonucleotides to different discrete areas in order to capture, e.g., differently oligonucleotide-tagged ligands thereto. This will permit different ligand conjugates to be addressed to different areas, but the capture surface may still be regenerated by general regeneration conditions.

Usually, the oligonucleotides have a length of at least six bases. It is further preferred that the oligonucleotide pairs are completely complementary over at least a portion of their respective sequences. Completely complementary sequences are, however, preferred.

Heterobifinctional agents that may be used to prepare ligand/oligonucleotide conjugates, such as, e.g., antibody/oligonucleotide conjugates, are well known to and may readily be selected by the skilled person. For examples of such heterobifunctional agents, it may be referred to, for instance, the above-mentioned U.S. Pat. No. 5,648,213 (the disclosure of which is incorporated by reference herein).

A reagent kit for providing a solid support surface with ligand attached via an oligonucleotide duplex may comprise a first oligonucleotide having a function for coupling to a solid support, a second oligonucleotide complementary to the first oligonucleotide and having a function for direct or indirect coupling to a ligand, and a surfactant. Both the first and second oligonucleotides may, for example, be aminonucleotides. The aminonucleotide to be coupled (such as conjugated) to a ligand may be provided in the kit either prepared for direct coupling, or in such a form that it may easily be activated and coupled to a ligand having a suitable function for reaction with the activated group. For example, an aminonucleotide may be indirectly thiolated by reaction with N-succinimidyl 3-(2-pyridyldithio)propionate, followed by reduction of the 3-(2-pyridyldithio)propionyl conjugate with dithiothreitol (DTT) or tris-(2-carboxyethyl) phosphine (TCEP). The thiol function may then be reacted with a ligand modified with a thiol-reactive group, such as a maleimide group. The latter may, for instance, be introduced at an amine site using succinimidyl trans-4-(maleimidylmethyl)cyclohexane-1-carboxylate.

The reagent kit may include instructions for use, e.g., in the form of a label on a package containing the kit ingredients, or on a package insert. Such instructions may include inter alia information on how to mix the surfactant with an aqueous liquid, e.g., a buffer, to form a vesicular structure, usually a micelle or a liposome.

Examples of analytes that may be assayed for include, without any restriction thereto, agonists and antagonists for cell membrane receptors, toxins and venoms, viral epitopes, hormones (e.g., opiates, steroids, etc), hormone receptors, peptides, enzymes, enzyme substrates, cofactors, drugs, lectins, sugars, oligonucleotides, oligosaccharides, proteins, and monoclonal antibodies. Assaying an analyte is not restricted to qualitative or quantitative determination of the analyte, but also includes, for example, studying its interaction with a ligand for other characterization of the analyte, such as determining binding properties, e.g., affinity and kinetic constants.

Methods for detecting the presence of bound analyte(s) on the surface may be chosen from a wide variety of detection techniques, including both photometric and non-photometric methods of detection, for example, marker-based techniques, where the analyte(s) or an analyte specific reagent(s) is (are) labelled, e.g., with a radiolabel, a chromophore, fluorophore, chemiluminescent moiety or a transition metal; as well as label-free techniques.

For many applications, the assays are performed with a biosensor. Biosensors may be based on a variety of detection methods. Typically such methods include, but are not limited to, mass detection methods, such as piezoelectric, optical, thermo-optical and surface acoustic wave (SAW) device methods, and electrochemical methods, such as potentiometric, conductometric, amperometric and capacitance methods. With regard to optical detection methods, representative methods include those that detect mass surface concentration, such as reflection-optical methods, including both internal and external reflection methods, angle, wavelength or phase resolved, for example ellipsometry and evanescent wave spectroscopy (EWS), the latter including surface plasmon resonance (SPR) spectroscopy, Brewster angle refractometry, critical angle refractometry, frustrated total reflection (FTR), evanescent wave ellipsometry, scattered total internal reflection (STIR), optical wave guide sensors, evanescent wave-based imaging such as critical angle resolved imaging, Brewster angle resolved imaging, SPR angle resolved imaging, and the like. Further, photometric methods based on, for example, evanescent fluorescence (TIRF) and phosphorescence may also be employed, as well as waveguide interferometers. Also atomic force microscopy (AFR)-based detection methods may be mentioned.

In the Examples below, a biosensor instrument based on surface plasmon resonance (SPR) detection at a gold surface was used, providing “real-time” binding interaction analysis between a surface bound ligand and an analyte of interest by mass-sensing at the surface. A detailed discussion of the technical aspects of this type of biosensor, as well as of the phenomenon of SPR, may be found in the above-mentioned U.S. Pat. No. 5,313,264. More detailed information on matrix coatings for biosensor sensing surfaces is given in, for example, U.S. Pat. Nos. 5,242,828 and 5,436,161. In addition, a detailed discussion of the technical aspects of the biosensor chips used with the biosensor instrument may be found in the above-mentioned U.S. Pat. No. 5,492,840. (The full disclosures of the above patents are incorporated by reference herein).

In the following Examples, various aspects of the present invention are disclosed more specifically for purposes of illustration and not limitation.

### EXAMPLES

A BIACORE® 3000 instrument (Biacore AB, Uppsala, Sweden) was used. In this instrument, a micro-fluidic system passes samples and running buffer through four individually detected flow cells (one by one or in series), with very high precision and with small sample volumes needed. As sensor chip was used Sensor Chip CM5 (Biacore AB, Uppsala, Sweden) which has a gold surface with a covalently linked carboxymethyl-modified dextran polymer hydrogel. Running buffer was HBS-N (10 mM HEPES pH 7.4 and 150 mM NaCl) (Biacore AB, Uppsala, Sweden). Due to the carboxy groups, the hydrogel has an anionic character. The output from the instrument is a “sensorgram” which is a plot of detector response (measured in “resonance units”, RU) as a function of time. An increase of 1000 RU corresponds to an increase of mass on the sensor surface of approximately 1 ng/mm².

The following two (complementary) oligonucleotides were used (chemically modified at the 5′-end with an amino group):

5′TTT CCT CAG CAT CTT ATC CG3′, referred to as “BC1”

5′CGG ATA AGA TGC TGA GGA AA3′, referred to as “BC2”

The nucleotide sequence BC1 is disclosed in Persson, B., et al. (1997) Anal. Biochem. 246, 34-44.

### Example 1 (Comparative)

### Immobilization of Amino-Modified Oligonucleotide BC1 at High Salt Concentrations

Sensor Chip CM5 was activated by 0.2 M N-ethyl-N-dimethylamino-proplycarboiimide (EDC) and 50 mM N-hydroxysuccinimide (NHS) for 7 or 20 minutes at a flow of 5 μl/min (EDC and NHS were from Biacore AB, Uppsala, Sweden), converting a fraction of the carboxyl groups on the dextran to reactive N-hydroxysuccinimde ester groups. 100 μM of amine-modified oligonucleotide BC1 (SGS DNA, Köping, Sweden, or DNA Technology, Ålborg, Denmark) in borate 8.5 immobilization buffer (Biacore AB, Uppsala, Sweden) was then injected for 13 minutes at 5 μl/min for immobilization thereof to the surface. Unreacted N-hydroxysuccinimide ester groups were deactivated by injecting ethanolamine for 3 minutes at 5 μl/min (replacing the activated group by hydroxyethylamide). The immobilization procedure was performed at high NaCl concentrations varying from 1 to 3 M to shield off the negative charges of the oligonucleotide, and at relatively high pH values between 7.0 and 8.5 to obtain a rapid coupling to the surface.

After each immobilization, the surface was washed with three pulses of 50 mM NaOH, 1 M NaCl for 1 min (flow 5 μl/min) to remove loosely bound oligonucleotide. The complementary amino-modified oligonucleotide BC2 as well as a non-complementary oligonucleotide (BC1) were then allowed to hybridise to the immobilized amino-nucleotide BC1 for three minutes at a flow of 5 μl/min with HBS-EP buffer (Biacore AB, Uppsala, Sweden). Oligonucleotide concentrations varied between 1 and 10 μM. Regeneration of the surface between hybridizations was performed with 50 mM NaOH, 1 M NaCl for 1 minute.

The results are shown in Table 1 below.

TABLE 1

| | | | | Hybridization | Hybridization |
|---|---|---|---|---|---|
| | NaCl | NHS/EDC | | of comple- | non- |
| | conc. | Activation | Immobilization | mentary | complementary |
| pH | (M) | time (min) | (RU) | oligo (RU) | oligo (RU) |
| 7.0 | 1 | 7 | −26 | 9.7 | 4.8 |
| 7.0 | 3 | 7 | −5.4 | 1.9 | 7.5 |
| 8.5 | 1 | 7 | 16.5 | 8.6 | 6.9 |
| 8.5 | 3 | 7 | 40.8 | 10.1 | 6.8 |
| 7.0 | 1 | 20 | 74.2 | 11.6 | 10.1 |
| 7.0 | 3 | 20 | −28.9 | 3.2 | 8.4 |
| 8.5 | 1 | 20 | −7.8 | 3.7 | 7.3 |
| 8.5 | 3 | 20 | 15.0 | 4.4 | — |

As appears from the Table, a high salt concentration was not effective to increase immobilization of the amino-oligonucleotide to the surface. Neither was the immobilization influenced by varying the pH or increasing the NHS/EDC activation time.

### Example 2 (Comparative)

### Immobilization of Amino-Modified Oligonucleotide BC1 with Presence of Tetramethylammonium Chloride

Following the same immobilization protocol as in Example 1, oligonucleotide BC 1 was immobilized to a CM5 chip surface, except that HBS-EP (Biacore AB, Uppsala, Sweden) was used as immobilization buffer, and 1M, 0.75 M, 0.5 M or 0.25 M tetramethylammonium chloride (TMA-Cl) was used instead of NaCl to shield off the negative charges of the oligonucleotide. After washing with 50 mM NaOH, 1 M NaCl, hybridizations with complementary and non-complementary oligonucleotides were then performed as described in Example 1. The results are shown in Table 2 below.

TABLE 2

| | | Hybridization of | Hybridization of |
|---|---|---|---|
| TMA-Cl | Immobilization | complementary | non-complementary |
| conc (M) | (RU) | oligo (RU) | oligo (RU) |
| 0.25 | −0.9 | 6.7 | 4.5 |
| 0.50 | −4.1 | 3.8 | 4.9 |
| 0.75 | −1.7 | 3.5 | 4.8 |
| 1.00 | 3.5 | 3.3 | 4.3 |

As appears from the Table, TMA-Cl had no effect on the immobilization of oligonucleotide to the surface.

### Example 3

### Immobilization of Amino-Modified Oligonucleotide BC1 Through Complexing with Cetyltrimethylammonium Bromide Micelles

Sensor Chip CM5 was activated as described in Example 1 above. 10-50 μM of amine-modified oligonucleotide BC1 (SGS DNA, Köping, Sweden, or DNA Technology, Ålborg, Denmark) in 10 mM Hepes pH 7.4 (Biacore AB, Uppsala, Sweden) with varying concentrations of cetyltrimethylammonium bromide (CTAB) were then injected for 10 minutes at 5 μl/min for immobilization thereof to the surface. Unreacted N-hydroxysuccinimide ester groups were deactivated by injecting ethanolamine for 3 minutes at 5 μl/min. After washing with 50 mM NaOH, 1 M NaCl, hybridizations with complementary and non-complementary oligonucleotides were then performed as described in Example 1. The results are shown in Table 3 below.

TABLE 3

| | | | | Hybridization | Hybridization |
|---|---|---|---|---|---|
| | Amino- | | | of comple- | of non- |
| CTAB | oligo conc. | Immobilization | Loss at | mentary | complementary |
| conc. (mM) | (μM) | (RU) | wash (RU) | oligo | oligo |
| 0.40 | 10 | 3170 | −690 | 2181 | −0.6 |
| 0.60 | 10 | 3440 | −649 | 2701 | 2.0 |
| 0.80 | 10 | 3322 | −612 | 2390 | 1.8 |
| 1.00 | 10 | 2763 | −527 | 1877 | 0.8 |
| 0.75 | 25 | 3075 | −423 | 1569 | −1.3 |
| 1.50 | 25 | 3175 | −474 | 2375 | 2.1 |
| 2.25 | 25 | 3170 | −500 | 2317 | 2.3 |
| 3.00 | 25 | 3046 | −511 | 2258 | 2.2 |
| 2.25 | 50 | 3121 | −511 | 2388 | −1.5 |
| 3.00 | 50 | 3147 | −471 | 2349 | 1.4 |
| 3.75 | 50 | 3103 | −442 | 2312 | 2.5 |
| 4.50 | 50 | 2863 | −446 | 2242 | 2.8 |

As can be seen in the Table, high immobilization and hybridisation levels were obtained. The most suitable concentration of CTAB apparently varies with the concentration of the oligonucleotide BC1. The critical micellar concentration (CMC) for CTAB in the immoblization buffer used is likely to be near 0.29 mM. Since an average micelle of CTAB contains 61 molecules, the Table indicates an optimum micelle:oligo ratio of approximately 1:1 (0.60 mM CTAB/10 μM BC1; 1.50 mM CTAB/25 μM BC1; and 3.00 mM CTAB/50 μM BC1, respectively).

### Example 4

### Immobilization of Amino-Modified Oligonucleotide BC1 Through Complexing with Dodecyltrimethylammonium Bromide Micelles

The procedure described in Example 3 was followed, except that dodecyltrimethylammonium bromide (DTAB) was substituted for cetyltrimethylammonium bromide (CTAB). No hybridization with non-complementary oligonucleotide was performed. The results obtained are shown in Table 4 below.

TABLE 4

| | | | Loss at | Hybridization |
|---|---|---|---|---|
| DTAB conc. | Amino-oligo | Immobilization | wash | of comple- |
| (mM) | conc. (μM) | (RU) | (RU) | mentary oligo |
| 0 | 10 | 10.5 | −116.6 | 15 |
| 5 | 10 | 3054.5 | −625.8 | 1970.7 |
| 10 | 10 | 3372.7 | −680.4 | 2054.1 |
| 15 | 10 | 3519.7 | −625.4 | 2276.5 |

From the foregoing, it will be appreciated that, although specific embodiments of this invention have been described herein for purposes of illustration, various modifications may be made without departing from the spirit and scope of the invention. Accordingly, the invention is not limited except by the appended claims.

1. A method of immobilizing a target molecule to a solid support surface capable of interacting with the target molecule, which method comprises the steps of:
complexing the target molecule with a vesicular structure capable of forming a dissociable complex with the target molecule,
contacting the complex formed with the solid support surface to thereby bind the target molecule to the surface,
dissociating the complex, and
removing the vesicular structure from the solid support surface to leave the target molecule immobilized on the surface.

2. The method according to claim 1, wherein the vesicular structure is selected from liposomes and micelles.

3. The method according to claim 1, wherein the vesicular structure is a micelle.

4. The method according to claim 1, wherein the target molecule and the vesicular structure carry opposite electric charges.

5. The method according to claim 1, wherein the target molecule and the solid support surface carry electric charges of the same kind.

6. The method according to claim 1, wherein the target molecule carries a negative charge.

7. The method according to claim 1, wherein the target molecule and the solid support surface each carry a negative charge, and the vesicular structure carries a positive charge.

8. The method according to claim 1, wherein binding of the target molecule to the surface causes at least partial dissociation of the complex.

9. The method according to claim 1, wherein the target molecule is a ligand capable of binding an analyte.

10. The method according to claim 1, wherein the target molecule is a capture agent capable of binding a ligand or a ligand-binding agent.

11. The method according to claim 1, wherein the target molecule is selected from nucleic acids and antibodies.

12. The method according to claim 11, wherein the target molecule is an oligonucleotide.

13. The method according to claim 11, wherein the target molecule is an artificial oligonucleotide.

14. The method according to claim 1, wherein the target molecule is a low molecular weight organic compound.

15. The method according to claim 1, wherein the solid support surface comprises a reactive group capable of reacting with a functional group of the target molecule to form a covalent bond.

16. The method according claim 1, wherein the solid support surface comprises one member of a specific binding pair, and the other member of the binding pair is conjugated to or part of the target molecule.

17. The method of claim 16, wherein the surface-bound member is avidin or streptavidin, and the target molecule is biotin-tagged.

18. The method according to claim 1, wherein the solid support surface comprises a hydrogel.

19. The method according to claim 18, wherein the hydrogel is based on dextran.

20. The method according to claim 19, wherein the dextran comprises carboxymethyl groups.

21. The method according to claim 20, wherein the carboxymethyl groups are activated to reactive groups.

22. The method according to claim 1, wherein the ratio of target molecule to vesicular structure is about 1:1.

23. The method according to claim 3, wherein the ratio of target molecule to micelle is about 1:1.

24. The method according to claim 1, wherein the vesicular structure is a micelle comprising cetyltrimethylammonium bromide (CTAB).

25. The method according to claim 1, wherein the method is carried out in a flow cell.

26. The method according to claim 1, wherein the solid support is a sensor surface.

27. The method according to claim 26, wherein the sensor surface permits detection of events at the surface by mass-sensing.

28. The method according to claim 27, wherein the mass-sensing comprises evanescent wave sensing.

29. The method according to claim 28, wherein the evanescent wave sensing is based on surface plasmon resonance.

30. The method according to claim 1, wherein the solid support is a chromatographic particle.

31. A method of sensitizing a solid support surface with a ligand, which method comprises the steps of:
providing a capture agent for the ligand, which capture agent is capable of binding to the solid support surface,
complexing the capture agent with a vesicular structure capable of forming a dissociable complex with the capture agent,
contacting the complex formed with the solid support surface to thereby bind the capture agent to the surface,
dissociating the complex,
removing the vesicular structure from the solid support surface to leave the ligand immobilized on the surface, and
contacting the solid support surface with the ligand to bind the ligand to the immobilized capture agent.

32. The method according to claim 31, wherein the capture agent is an oligonucleotide, and the ligand is conjugated to an oligonucleotide complementary to the capture oligonucleotide.

33. The method according to claim 31, wherein different discrete areas of the solid support surface supporting a general capture agent are selectively contacted with different ligands to provide a solid support surface with an array of different ligands.

34. The method according to claim 31, wherein different discrete areas of the solid support surface, each supporting a different capture agent, are contacted with different ligands to provide a solid support surface with an array of different ligands.

35. The method according to claim 31, wherein the solid support surface is a sensor surface.

36. A method for assaying a sample for at least one analyte, which method comprises contacting the sample with a solid support surface sensitized with at least one analyte-binding ligand by to the method according to claim 31, and detecting binding of the analyte to the surface.

37. A method for studying analyte-ligand binding interactions, which method comprises contacting at least one analyte with a solid support surface sensitized with at least one analyte-binding ligand by to the method according to claim 31, and studying binding interactions between anlyte and ligand at the surface.

38. A reagent kit comprising:
a first oligonucleotide having a function for coupling to a solid support,
a second oligonucleotide complementary to the first oligonucleotide and having a function for direct or indirect coupling to a ligand, and
a surfactant.

39. The kit according to claim 38, wherein the first and second oligonucleotides independently of each other are selected from aminoligonucleotides.

40. The kit according to claim 38, wherein the second oligonucleotide is an aminoligonucleotide modified by N-succinimidyl 3-(2-pyridyldithio)propionate (SPDP) conjugation.

41. The kit according to claim 40, wherein the kit further comprises a reagent for reducing the pyridyldithio group of the N-succinimidyl 3-(2-pyridyldithio)propionate-modified aminoligonucleotide to a thiol group.

42. The kit according to claim 38, wherein the surfactant is cetyltrimethylammonium bromide (CTAB).

43. The kit according to claim 38, wherein the kit further comprises instructions for use thereof.

44. The kit according to claim 43, wherein the instructions comprise directions for mixing the surfactant with an aqueous liquid such that the surfactant forms vesicular structures in the liquid.
